(19) United States
(12) Patent Application Publication
(10) Pub. No.: US 2011/0166347 A1
(43) Pub. Date: Jul. 7, 2011

(54) PROCESS FOR PREPARATION OF MYCOPHENOLIC ACID, ITS SALT AND ESTER DERIVATIVES

(75) Inventors: Ashok Kumar, Mumbai (IN); Dharmendra Singh, Mumbai (IN); Umesh Luthara, Mumbai (IN); Yogesh Mohandhai Patel, Mumbai (IN)

(73) Assignee: IPCA Laboratories Ltd., Mumbai (IN)

(21) Appl. No.: 13/062,416

(22) PCT Filed: Sep. 9, 2009
(86) PCT No.: PCT/IN2009/000493

§ 371 Date: Mar. 4, 2011

(30) Foreign Application Priority Data
Sep. 10, 2008 (IN) 1921/MUM/2008

(51) Int. Cl. C07D 413/12; C12P 17/04; C07D 307/88

(52) U.S. Cl. 544/153; 435/126; 549/305

(57) ABSTRACT

The present invention discloses an isolation and purification process for mycophenolic acid obtained from the fermentation process. Invention further discloses preparation of sodium salt of mycophenolic acid and mycophenolate mofetil from mycophenolic acid.

### FIELD OF INVENTION

The present invention relates to an isolation and purification process for mycophenolic acid obtained from the fermentation process. Invention further relates to preparation of sodium salt of mycophenolic acid and mycophenolate mofetil from mycophenolic acid.

### BACKGROUND OF THE INVENTION

Mycophenolic acid, chemically known as 6-[4-hydroxy-6-methoxy-7-methyl-3-oxo-5-phthalanyl]-4-methyl-4-hexanoic acid (I) was discovered & isolated in 1893, as a secondary metabolite of fungus of penicillium genus.

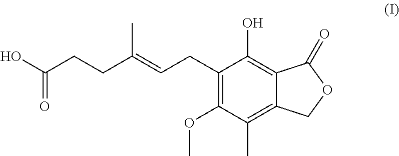

Market approved dosage forms contained of sodium salt of mycophenolic acid as well as mycophenlate mofetil. Preparative method for Sodium salt of mycophenolic acid (II) was disclosed in ZA6804959

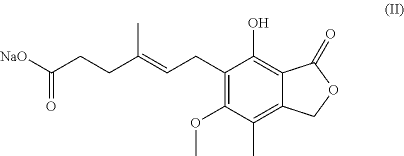

and for ester derivative mycophenlate mofetil (III) was disclosed in 1986.

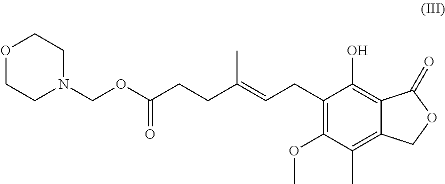

Mycophenolic acid or its salts as well as esters possesses various pharmaceutically valuable activities such as antibiotic, antivral and anti-tumor properties and it is a powerful immunosuppressant. Sodium mycophenolate and mycophenolate mofetil are clinically approved for treatment of psoriasis and as an immunosuppressant agent for the prevention of rejection in organ transplant.

Various prior arts such as GB 1158387, GB 1157099, U.S. Pat. No. 4,115,197, U.S. Pat. No. 4,452,891, WO2008/002665, WO01/21607, EP1624070 disclose process for preparation and isolation of mycophenolic acid. Among several species of Penicillium identified to produce Mycophenolic acid, Penicillium Brevicompactum is widely used for the industrial production. All these fermentation processes involves submerged or solid state fermentation either batch mode or fed batch. Better conversions were reported with fed batch mode of fermentation and submerged culturing is preferred as uniform mixing and feeding or aeration can be easily achieved. Several processes include use of mutating agents to improve yield. However, the reported yields from all these processes are very low i.e. in the range of 1.3 mg/litre to 363 mg/litre.

Solid state fermentation is reported by Sadhukaran et al, in J. ind. Micro. Biotechnology, (year??) resulting into a yield of 3.286 g MPA/Kg biomass.

WO2008/002665 uses mutated strain and reports higher production rate but this is claimed to be achieved by maintaining oxygen level at a specified rate during the fermentation process.

Methods for isolation and purification of mycophenolic acid from fermentation broth are known. An improved method for purification of mycophenolic acid by using pH adjustment and extraction of whole broth or medium with water immiscible solvents is reported in WO2005/105768, wherein 119.4 Kg concentrated mycophenolic acid suspension provides only 553 gm of mycophenolic acid.

Although there are synthetic methods reported for mycophenolic acid production, however, a biological fermentation method is most commonly used.

The yield of mycophenolic acid in fermentation methods reportedly depends on several parameters like fermentation conditions; feed character and fed rate, and ultimately the type of the strain used. Though all other parameters are critical, the type of strain is the most crucial for getting higher production. It is also known that wild strain gives only small yields of mycophenolic acid and procedures are needed to improve efficiency of wild strains of Penicillium.

A major technical difficulty of the fermentation process is the isolation of mycophenolic acid from the fermentation broth containing such a low concentration of the active compound mycophenolic acid. Generally the isolation comprises pH adjustment of the fermentation broth to either acidic or alkaline and extraction of the whole broth with a water immiscible organic solvent with or without elimination of mycelia, but it recovers very small amount of mycophenolic acid from the broth.

For the economy of the process, the mycophenolic acid should be recovered completely from the fermentation broth, which is almost impractical in reported aqueous processes because of poor efficiency of extraction from large volume of aqueous solutions. In most cases very large volume of organic solvent are required for recovering a small amount of mycophenolic acid, which is not economically viable.

Mycophenolic acid can be converted to its sodium salt which is known as sodium mycophenolate or mycophenolate sodium. Preparation of mono and di sodium salt of mycophenolic acid is disclosed in ZA 684959. Mono sodium salt (sodium mycophenolate) is prepared by reacting mycophenolic acid with sodium methoxide in presence of chloroform. Sodium mycophenolate obtained by this process is designated as Form M2, whose character is disclosed in US2006/0069152. Though many polymorphic forms are known, M2 form is preferred.

WO2006012379 discloses various processes for preparing sodium mycophenolate Form M2 starting either with mycophenolic acid or sodium mycophenolate itself. The process involves dissolving mycophenolic acid in selected solvents followed by treatment with sodium source and isolating the resultant sodium mycophenolate Form M2 either directly from solvent or by adding a precipitating solvent. If sodium mycophenolate is used as starting material it is processed in selected solvents to obtain Form M2. The specific solvents used in the process of '379 patent are selected from alcohols, alkyl esters etc.

All these processes report use of very large volumes of solvents which are difficult to handle on industrial scale and their removal by distillation takes up considerable energy and resources. The volumes also affect the production cycle as the output of every cycle will be very less as compared to the employed volumes in addition to this recycling or recovery of the solvent is also required to avoid the wastage and searching for solvents either be easily recoverable or the volume requirement is lower for recovery of required M2 form.

Mycofenolate Mofetil is disclosed in U.S. Pat. No. 4,753,935. The product obtained by process of '935 contains high percentage of dimeric impurity. Several improved processes disclosed in U.S. Pat. No. 5,247,083, WO0034503, WO02100855 involves direct esterification of mycophenolic acid. In general, 2-(4-morpholinyl)ethanol is used for direct esterification of mycophenolic acid and WO2009/084008 reports use of metal derivative of 2-(4-morpholinyl)ethanol. All these processes suffer from disadvantage of incomplete reaction, longer reaction periods besides generation of colored impurity. Attempts to achieve complete reaction using conventional methods only increases the impurity generation.

WO2004089946 reports use of microwave irradiation to complete the reaction, however such method lacks industrial applicability and costly to perform on large scale. Use of Enzyme to catalyze the reaction are reported in WO2003042393, WO 2000034503 and WO2006024582 but enzymatic reactions involve large volumes of biomass, hence are inconvenient on industrial scale. Transesterification is reported in US2004/0167130 but it requires use of toxic reagent such as dibutyl tin oxide to achieve required transesterification.

Apparently long reaction time, incomplete reaction and formation/contamination of colored impurities are the major difficulties in preparation of mycophenolate mofetil. To overcome one or more insufficiencies of prior art processes the present invention aims to provide modified processes for preparation of mycophenolic acid, its sodium salt and ester derivative i.e. mycophenolate mofetil. It is also an objective of the present invention to provide high mycophenolic acid producing mutant strains and an efficient process to recover the mycophenolic acid from the fermentation broth, apart from simplifying the process for purification of mycophenolic acid. The present invention ameliorates problems in the prior art and also reduced a number of unit operations of pH assisted extraction/back extractions.

### SUMMARY OF THE INVENTION

Accordingly, the present invention provides a process for producing mycophenolic acid and its recovery from the fermentation broth. Invention also provides improved process for conversion of mycophenolic acid into sodium mycophenolate and Mycophenolate Mofetil.

According to one aspect of the present invention, a process for mycophenolic acid is provided comprising the steps of:
a) culturing a mycophenolic acid producing microorganism under suitable conditions;
b) adjusting the pH of the fermentation culture obtained in step a) to acidic;
c) recovering the mycelia by filtration or other means;
d) leaching the mycelia with an organic solvent; and
e) isolating the mycophenolic acid from the solvent; and, optionally
f) Purifying the mycophenolic acid obtained.

The microorganism is selected from a penicillium species of P. brevi-compactum, P. scabrum, P. nagemi, P. roqueforti, P. patris-mei and P. viridictum, preferably is Penicillium brevi-compactum. Mutated version of the high producing Penicillium species is used, and mutated penicillium bervi-compactum is especially preferred. The pH of the fermentation broth is adjusted to 4.5 preferably below 3 before recovering the mycelia from the fermentation broth by filtration or other means.

In a second aspect, the present invention provides a mutated high producing strain of P. brevicompactum for the production of mycophenolic acid. The mutated strain is produced by treatment of wild P. Brevicompactum with N-methyl-N′-nitro-N-nitroso-guanidine, ethyl methyl sulfonate, clofibrate and methyl violgen

In a further aspect of the present invention, a process for recovery of mycophenolic acid from the fermentation broth is provided comprising the steps of:
a) adjusting the pH of the whole fermentation broth containing mycophenolic acid to acidic;
b) filtering out the mycelia;
c) leaching the mycelia with an organic solvent; and
d) Isolating mycophenolic acid from the organic solvent.

The organic solvents used for leaching includes solvents such as, but not limited to, hydrocarbons, esters, ketones, alcohols, protic solvents, aprotic dipolar solvents, ether and their mixtures thereof.

In a third aspect of the present invention, there is provided a purification method for mycofenolic acid comprising the steps of:
a) forming a solution of mycophenolic acid in an alkaline aqueous solution;
b) optionally, filtering the obtained solution; and
c) precipitating mycophenolic acid from the aqueous solution of step a) or b) by mixing with an acid.

In a further aspect, the invention provides a process for preparation of Sodium Mycophenolate and isolating crystalline Form M2, which comprises:
a. dissolving mycophenolic acid in a solvent selected from dimethyl sulfoxide dimethyl acetamide, N-methylpyrrolidone, acetonitrile, sulfolane or mixture thereof;
b. adding a source of sodium ion; and
c. isolating sodium mycophenolate form M2 from the reaction solution.

The source of sodium ion includes, but not limited to, sodium hydroxide, sodium methoxide and sodium ethoxide.

In a still further aspect, the invention provides a process for conversion of mycophenolic acid to mycophenolate mofetil.

In one embodiment, the process comprises reacting mycophenolic acid or its alkali salt with compound of formula (IV) or its salts and isolating mycophenolate mofetil.

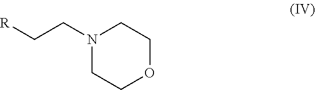

In the formula IV, R refers to a leaving group, especially Cl, Br, or a tosyl group and the like.

In another embodiment, the mycophenolate mofetil, according to the invention, is prepared by reacting mycophenolic acid with 2-(4-morpholinyl)ethanol in presence of molecular sieves.

The details of one or more embodiments of the inventions are set forth in the description below. Other features, objects and advantages of the inventions will be apparent from the appended examples and claims.

### DETAILED DESCRIPTION OF THE INVENTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The term “treating” means adding or combining or mixing the stated reagent or materials to the thing being treated.

The term “aqueous medium” means a solvent medium that does contain water in significant amounts. The term does not exclude water containing reasonable amounts of water miscible organic solvent, which may be less than 30%, more preferably less than 5%.

The term “leaching” includes without limitation any means of recovering the product from a solid mass with a solvent that commonly understood in the art, and includes extraction, infusion or steeping

It has now been found that a new process makes it possible to obtain mycophenolic acid in high yield and purity, ensuring the near complete recovery of mycophenolic acid from dilute fermentation broth media. The various aspects of the invention are described in detail with the specific embodiments/conditions hereafter.

Accordingly, in one aspect of the present invention, mycophenolic acid is prepared by a process comprising the steps of:
a) culturing a mycophenolic acid producing microorganism under suitable fermentation conditions;
b) adjusting the pH of the fermentation culture obtained in step a) to acidic;
c) isolating the mycelia from the fermentation broth/medium by filtration or other suitable means;
d) leaching the mycelia with an organic solvent to recover mycophenolic acid;
e) isolating the mycophenolic acid from the organic solvent; and optionally
g) purifying the resulting mycophenolic acid.

The microorganism for the production of mycophenolic acid is selected from any penicillium species capable of producing mycophenolic acid, the known strains includes P. brevi-compactum, P. scabrum, P. nagemi, P. roqueforti, P. patris-mei and P. viridictum. The most preferred strain is Penicillium brevi-compactum.

The yield of mycophenolic acid in the fermentation medium with the wild strain is very poor. Thus the present invention provides a mutated version of the high producing Penicillium species, especially P. brevi-compactum for increasing the production yield of mycophenolic acid in the broth. With the use of the mutated form of penicillium bervicompactum strain the production yield reaches more than 4.5 g-6 gm/litre in the fermentation medium compared to 1.3 gm/l with the use of wild strain. The high producing mutated strain of Penicillium brevi-compactum is obtained by treating the wild strain with chemical agents to increase the potency for mycophenolic acid. The chemical agent used in the present invention is N-methyl-N′-nitro-N-nitroso-guanidine, ethyl methyl sulfonate, clofibrate, and methyl viologen.

The culturing of the organism may be done according to any known method including either submerged or solid state with or without a fed-batch mode of feeding the organism. The most preferred method is submerged culturing with a fed-batch mode of feeding the organism in the production fermentation stage. The nutrient feed may contain the materials known per se for good growth of the organism, including carbon and nitrogen sources, buffers, and growth enzymes.

Exemplarily carbon source includes, but not limited to, glucose, sucrose, starch, maltose, malto-dextrin, malt, cotton seal meal, soyabean oil meal etc. Exemplarily nitrogen source material may be used in the fermentation process includes, known amino acids, ammonium salts like ammonium nitrate & ammonium nitrate, nitrates like sodium nitrate, bacteriological peptone, yeast extract, casein hydrolyzate, soya peptone, glycine, seed meals, for example cottonseed meal, and corn steep liquor, peptone, urea, yeast extract and meat extract. For the maintenance of pH of the fermentation medium, known buffers are added which are customarily used for this application, including phosphate buffer), the source of phosphorous, sulphur and potassium may be for example potassium dihydrogen phosphate, a soluble suphate and potassium chloride respectively. The pH of the medium is maintained during the course of fermentation in the range between 6.0 to 7.5. The fermentation medium usually comprised of aqueous solvent, especially water. Sufficient aeration may be given for maintenance of the oxygen level

The fermentation is carried out usually at a temperature not lower than ambient temperature and not higher than the temperature for the survival of the strain. Preferably the fermentation is carried out at a temperature from ambient temperature to 37 degrees. Especially preferred fermentation temperature is between 25-30 degree Celsius. The fermentation time is usually range from about 100 hours to about 4-16 days preferably 8-10 days.

The present inventors have found that the isolation of the mycophenolic acid from the fermentation medium according to the prior art processes present substantial difficulties due to the requirement of large volume of the organic solvents for recovering it from the medium and solubility of mycophenolic acid in both water and water immiscible solvents makes it even worse. The efficiency of the extraction is also limited due to very limited choices of organic solvents which has good solubility differences between aqueous and water immiscible solvents for better recovery of mycophenolic acid. The present inventors had recognized that in using water immiscible solvents for extraction of large volume of fermentation broth containing 1 gm/litre-5 gm/litre mycophenolic acid is not practical for the industrial application, owing to the economy of the process. Apart from this, repeated extraction of the fermentation medium might lead to contamination of the product with various organic impurities from the fermentation medium. The invention, therefore, aims to provide an improved method for quantitative recovery of mycophenolic acid from the fermentation broth/medium, and even makes it possible to use water miscible solvents for recovery of mycophenolic acid.

Thus the process for recovery of mycophenolic acid from the fermentation broth, according to the present invention, includes the steps of:
a) adjusting the pH of the whole fermentation broth containing mycophenolic acid to acidic
b) filtering out the mycelia;
c) leaching the mycelia with an organic solvent; and
d) isolating mycophenolic acid from the organic solvent.

The pH of the fermentation broth is adjusted to below 7, preferably about 4.5 or lower by means of an acid before isolating mycelia. The acid can be of organic or inorganic acid customarily used for acidification including, but not limited to, hydrochloric acid, sulfuric acid, phosphoric acid, methane sulfonic acid, acetic acid, formic acid etc.

In the process, after acidification of the fermentation broth, the mycelia are separated from the fermentation medium by any conventional means such as filtration or centrifugation. It has been found that the isolation & separation of mycelia leads to quantitative recovery of mycophenolic acid from the medium and better utilization of solvent for the recovery of mycophenolic acid as the mycelia (containing mycophenolic acid) quantity is only about ⅙^{th} of the total fermentation medium. The mycelia are leached with an organic solvent to recover the mycophenolic acid.

Suitable organic solvents for leaching the mycelia are those solvents possess reasonable solubility for mycophenolic acid, including, but not limited to, protic and aprotic polar solvent, and non-polar solvent. Exemplarily solvents include esters such as ethyl acetate, & butyl acetate, hydrocarbons such as toluene, cyclohexane, benzene etc., chlorinated hydrocarbons such as dichloromethane, ethylene dichloride, chloroform etc., ethers such as tetrahydrofuran, ketones such as acetone, methylethyl ketone, and alcohols such as isopropyl alcohol, butanol etc. Especially preferred solvent for this application is toluene and ethyl acetate or their mixtures thereof. The process of the present invention is characterized by use of small volume of organic solvent compared to very large volume of solvent required for extraction of mycophenolic acid from the whole fermentation medium.

The extraction/leaching of the mycelia may be carried out at ambient temperature or under heating or with hot organic solvents. Typically the leaching is done at room temperature; however, leaching with hot solvents may lead to further reduction in the solvent volume.

Mycophenolic acid is isolated from the organic solvent after leaching of the mycelia by any conventional means such as crystallization, evaporation, chromatography or any other means known to a skilled artisan. The preferred method includes partial evaporation of the organic solvent and crystallization of the mycophenolic acid from the concentrated solution. Alternately, mycophenolic acid can be recovered from the solution by addition of an anti-solvent to the concentrated solution obtained after leaching the mycelia. The anti-solvent for precipitation of mycophenolic acid from the extraction solvent include non-polar solvents such as hexane, heptane etc.

In another embodiment of the present invention, the mycophenolic acid may be recovered from the mycelia with use of aqueous solvent alone comprising adjusting the pH to alkaline with a base in aqueous solvent and filtering out the mycelia free of mycophenolic acid, followed by precipitating the mycophenolic acid from the aqueous solvent by adjusting the pH to acidic. The aqueous solvent is water. This makes the process to partially eliminate the use of organic solvent.

The purity of the mycophenolic acid exceeds 96% by assay in the methods of the present invention, with a recovery yield of greater than 95% based on the overall titer value of mycophenolic acid in the whole fermentation broth.

The mycophenolic acid may further purified by a novel method. The method is characterized by use of primarily aqueous solvent, especially water for purification which provides a pharmaceutically acceptable pure mycophenolic acid without the need for extractive methods as described in the art.

Thus, according to the present invention, there is provided a purification method for mycofenolic acid comprising the steps of:
a) forming a solution of mycophenolic acid in an alkaline aqueous solution;
b) filtering the obtained solution; and
c) precipitating mycophenolic acid from the aqueous solution in step b) by acidification.

The alkaline aqueous solution is made by dissolving appropriate quantity of alkali in water or else alkali may be added to a suspension of mycophenolic acid in aqueous solvent in order to form the solution of mycophenolic acid. The aqueous solvent is water. The alkali may be selected from any known base substance of organic or inorganic origin; however, inorganic bases are preferred for this application. Especially preferred inorganic base is sodium/potassium hydroxide or sodium/potassium carbonates/bicarbonates. The solution may be formed under heating or at room temperature, preferably under heating to about 30-50 degrees the alkaline aqueous solution of mycophenolic acid is optionally filtered to remove insolubles. The filtrate may be further subjected to active charcoal treatment to remove impurities, Mycophenolic acid can be precipitated by addition of acid to the filtrate. Typically the acid can be of organic or inorganic acid customarily used for acidification including, but not limited to, hydrochloric acid, sulfuric acid, phosphoric acid, methane sulfonic acid, acetic acid, formic acid etc. Preferably the acid is hydrochloric acid or sulfuric acid. The precipitation is carried out at ambient temperature or with appropriate cooling of the alkaline solution of mycophenolic acid. Once the precipitation/crystallization is completed the pure product may be isolated by conventional methods such as filtration, centrifugation, or similar unit operations as exemplified in the accompanying illustrative examples.

The mycophenolic acid can be further converted to sodium mycophenolate Form M2. Thus, according to the present invention; there is provided a process for preparation of sodium mycophenolate Form M2 comprising the steps of
a. dissolving mycophenolic acid in a solvent selected from N-methylpyrrolidone, sulfolane, dimethyl acetamide, acetonitrile and their mixture thereof;
b. adding a source of sodium ion;
c. precipitating sodium mycophenolate; and
d. isolating sodium mycophenolate Form M2.

Mycophenolic acid used in the process can be obtained by process of present invention or by any method known in the art. The process includes dissolving mycophenolic acid in solvent disclosed in step a, followed by addition of source of sodium ion, once the clear solution is obtained the reaction mixture is stirred at ambient temperature. An anti-solvent can be added to precipitate sodium mycophenolate.

Any source of sodium ion may be used in this process, for example sodium hydroxide, sodium methoxide, sodium ethoxide or like. However, the preferred are sodium methoxide and hydroxide. Usually the reaction is carried out at room temperature but can be practiced at higher or lower temperature. The anti-solvent may be preferably selected from isoprapanol, n-pentane and n-hexane.

Surprisingly, it was found that the reaction can be carried out effectively with lower quantity of solvent. This reduces the total solvent consumption drastically moreover the product thus obtained passes through the residual solvent test as the solvents used in present process can be removed.

Mycophenolic acid or its salt can be further converted to mycophenolate mofetil. Thus according to the present invention there is provided an improved process for preparation of mycophenolate mofetil. In one embodiment, the process comprising the steps of
a. reacting mycophenolic acid or its salt with a compound of formula IV or its salt to obtain mycophenolate mofetil; and
b. isolating mycophenolate mofetil.

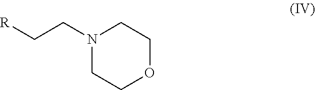

In the formula IV, R refers to a leaving group, especially Cl, Br, or a tosyl group and the like. It is preferable to use compound IV as salt, especially hydrochloride salt, as the preparation of chloroethinylmorpholine results in hydrochloride, and it can be directly used in the process.

The salt of mycophenolic acid can be selected from alkali or alkali earth metal salts, however the preferred salt is sodium mycophenolate. The sodium mycophenolate can be prepared according to process provided by present invention or by any method known in the art.

The reaction of mycophenolic acid or salt with compound of formula IV is preferably carried out in presence of solvent and a base. The preferred base is triethyl amine but any known base customarily used for this kind of reaction can be employed. The suitable solvent can be any organic or inorganic solvent, preferably polar solvents like dimethyl formamide, dimethyl sulphoxide etc. Reaction may be performed under heating, at about 40 to reflux temperature of solvent. The reaction gets completed in few hours as compared to long hours required by the reported processes. The colored impurity is completely absent in product prepared by present process. Hence this enables attainment of highly pure compound with a considerable reduction in reaction time.

In another embodiment mycophenolate mofetil is prepared by using molecular sieves in the reaction, and the process comprises steps of:
a. dissolving mycophenolic acid and 2-(4-morpholinyl)ethanol in a solvent;
b. heating the reaction mixture obtained in step (a) in presence of molecular sieves to obtain mycophenolate mofetil; and
c. isolating mycophenolate mofetil from the reaction mixture.

With the use of molecular sieves in the reaction, a better conversions of starting mycophenolic acid was achieved, unlike the reactions in its absence. The solvent used in step (a) can be any organic solvent like dimethyl formamide, dimethyl acetamide, N-methylpyrrolidone, dimethylsulphoxide, triethylamine, sulfolane etc. The reaction may be conducted under heating, preferably at a range of 80°-140° C. After completion of reaction molecular sieves are separated by filtration and mycophenolate mofetil may be isolated by an extractive work-up or other conventional techniques.

If desired, crude mycophenolate mofetil may be purified. The process of purification comprises:
a. dissolving crude mycophenolate mofetil in a solvent;
b. heating the reaction mixture to obtain a clear solution;
c. cooling the reaction mixture naturally;
d. separating the precipitated product by filtration.

The solvent for step (a) can be selected from any organic solvent, preferably n-butyl acetate is used. The reaction mixture is first cooled to the ambient temperature and then to 0-5° C.

The pure mycophenolic acid or its salt or its prodrug mofetil form obtained by the process of the present invention may be formulated into a dosage form, e.g., tablet, capsule, etc., by combining with one or more pharmaceutically acceptable excipients using known techniques. Further, the dosage form may be immediate release or extended release.

The examples provided below are illustrative and are not intended to limit the scope of the claimed invention.

### Example 1a

0.5 ml of spore suspension of mutated/modified P. brevicompactum strain was inoculated in 500 ml conical flask containing 60 ml of seed media. Inoculated media was incubated at 25° C., for 36 hrs. The seed culture was then loaded on to the bioreactor containing production media. Composition of production media was as follows:

| | | Quantity |
|---|---|---|
| Sr. No | Component | (gm/lit) |
| 1 | Sucrose | 48.0 |
| 2 | Cotton seed meal | 6.0 |
| 3 | Casein enzymatic | 9.0 |
| | hydrolysate | |
| 4 | KH2PO4 | 2.0 |
| 5 | MgSO4 | 0.1 |
| 6 | Polypropylene | 0.2 |
| | glycol | |
| 7 | Distilled water | 1000 ml |
| 8 | pH | 6.25 |
| 9 | Glycerol | |

The pH was maintained at 6.0 with sucrose feeding. The temperature was controlled at 25° C. for all 6 days, and 250 ml of soya oil and carbon source was added after completion of 146 hrs. Mycophenolic acid activity in the fermentation broth was 4.8 gm/liter.

### Example 1b

Example 1a was followed with a production media composition containing Sucrose, cotton seed meal, soyabean flour, Casein enzymatic hydrolysate, KH₂PO₄, MgSO₄, glycerol, propylene glycol, and water and a titre value of 5.0 gm of mycophenolic acid in the fermentation broth was obtained.

### Example 2

20 lit fermentation broth obtained by cultivation as per example 1, was acidified by addition of dil. HCl to pH 2.5 and the solution was stirred for 4-4.5 hrs at 30° C., and filtered. The residue 2-2.1 Kg (mycelia) was mixed with 6.6 liter of toluene and stirred for 2.0 hrs at 60-65° C. Solution was then filtered off; and the filtrate was concentrated to ⅓^{rd} volume. The solution was cooled to room temperature and stirred at the same for 30 minutes, and further cooled to 5-10° C. The precipitated mycophenolic acid was collected by filtration and the product was dried under vacuum. Yield was 90 gm and Purity was 90% by HPLC assay.

### Example 3

400 ml of water was added to the crude Mycophenolic acid (50 gm) obtained as per example 2, and aq. Solution of sodium carbonate (12.4 gm sodium carbonate dissolved in 100 ml water) was added. The resultant mixture was stirred for 1.5 hrs at ambient temperature. The solution was filtered. PH of the clear solution was adjusted to 2.5 with dilute HCl and the precipitated crystals were filtered and washed with water; the product was dried at 70° C. for 4-5 hrs to obtain 45 gm pure mycophenolic acid. Purity was 99%, by HPLC Assay.

### Example 4

20 ml of dimethylacetamide and 20 ml of methanol was added to 5 gm myophenolic acid and the resultant mixture was stirred for 10 minutes at ambient temperature. To the solution sodium methoxide Solution in methanol was added at ambient temperature. This reaction solution was added to 125 ml isopropyl alcohol to precipitate mycophenolate sodium salt in M2 form. The resultant mixture was filtered and precipitate washed with isopropyl alcohol to obtain sodium mycophenolate M2 form. Yield was 92% and purity was 99%.

### Example 5

100 ml of DMF and 2.2 gm of triethyl amine was added to 5.0 gm of mycophenolic acid at ambient temperature under stirring. To this mixture 3.2 gm of N-(2-chloroethyl)-morpholinium chloride was added. The reaction mixture was heated to 60-70° C. After completion of reaction, DMF was distilled off completely. 100 ml toluene was added to the residue, and the mixture was extracted with 20 ml of sodium carbonate (2.5%) aqueous solution. The organic layer was separated and mixed with 50 ml of water and pH adjusted to 2.5 by dil HCl. The aqueous layer was separated and basified. Resulting aqueous layer was extracted with toluene and the toluene layer was concentrated under vacuum to obtain 3 gm of mycophenoltate mofetil.

### Example 6

61.4 gm of 2-(4-morpholinyl)ethanol and 2.3 gm DMF was added to 100 gm Mycophenolic acid and the resultant mixture was heated. 14 gm molecular sieve was added to reaction mixture and it was heated at 120-130° C. for 20-24 hrs. The reaction mixture was cooled and 800 ml toluene was added. Molecular sieve were separated by filtration and to the resultant solution 400 ml sodium carbonate aqueous solution was added. Layers were separated and to the organic layer 600 ml water was added, the pH was adjusted to 2-2.5 by adding HCl and layers were separated and the pH of aqueous layer was adjusted to 8-8.5 by sodium carbonate. The precipitated product was extracted by toluene. The toluene was distilled off to obtain mycophenolate mofetil 100 gm, purity 99% by HPLC.

### Example 7

315 ml n-butyl acetate was added to 90 gm crude mofetil and heated to 70-75° C. until clear solution was obtained, and then cooled naturally to room temperature. The reaction mixture was further cooled to 0-5° C. and the precipitated product was filtered, and washed with n-butylacetate to obtain pure mycophenolate mofetil (yield: 79.4 gm and HPLC purity 99.5%.

### Example 8

10.0 g of mycophenlolic acid was mixed with 80 ml N-methylpyrrolidone in a flask. To this solution, aqueous sodium hydroxide solution (1.26 g of Sodium hydroxide in 18 ml of water) was added at 25-30° C. and stirred for 30 min. This reaction solution was added to 250 ml isopropyl alcohol dropwise at room temperature. The mixture was further stirred and then cooled to 10-15° C. and filtered it. Crystalline M2 form obtained was washed with isopropyl alcohol and dried. Dry weight=8.0 g.

### Example 9

10.0 g of mycophenlolic acid was mixed with 80 ml dimethyl acetamide in a flask. To this solution, aqueous sodium hydroxide solution (1.26 g of Sodium hydroxide in 18 ml of water) was added at 25-30° C. and stirred for 30 min. This reaction solution was added to 250 ml isopropyl alcohol dropwise at room temperature. The mixture was further stirred and then cooled to 10-15° C. and filtered it. Crystalline M2 form obtained was washed with isopropyl alcohol and dried. Dry weight=8.0 g.

1. A process for preparation of mycophenolic acid or its salt or derivatives comprising:
a) culturing a mycophenolic acid producing microorganism to produce a fermentation culture;
b) adjusting the pH of the fermentation culture obtained in step a) to acidic;
c) recovering the mycelia from the fermentation culture;
d) leaching the mycelia with an organic solvent or an alkaline solution to produce a leachate solution;
e) isolating mycophenolic acid from the leachate solution.

2. A process as claimed in claim 1, wherein the microorganism is selected from Penicillium species.

3. A process as claimed in claim 1, wherein the microorganism is penicillium brevicompactum.

4. A process as claimed in claim 1, wherein the microorganism is a mutated strain.

5. A process as claimed in claim 4, wherein the mutated strain is obtained by treatment with chemical agent.

6. A process as claimed in claim 1, wherein the pH is adjusted below 4.5.

7. A process as claimed in claim 1, wherein the pH is adjusted with organic or inorganic acid.

8. A process as claimed in claim 7, wherein the acid is selected from hydrochloric acid, sulfuric acid, phosphoric acid, acetic acid, formic acid and methane sulphonic acid.

9. A process as claimed in claim 1, wherein the organic solvent is a hydrocarbon, ester, ketone, alcohol, ether, protic solvents, aprotic solvents or mixtures there of.

10. A process as claimed in claim 1, wherein the organic solvent is ethyl acetate, butyl acetate, toluene, cyclohexane, benzene, dichloromethane, ethylene dichloride, chloroform, tetrahydrofuran, acetone, methylethylketone, isopropyl alcohol, butanol or mixture thereof.

11. A process as claimed in claim 1, wherein the step e) comprises concentrating the organic solvent; and precipitating mycophenolic acid.

12. A process as claimed in claim 1, wherein the alkaline solution comprises an organic or inorganic base.

13. A process as claimed in claim 1, further comprising purification of mycophenolic acid including the steps of:
a) forming a solution of mycophenolic acid in an alkaline aqueous solution; and
b) precipitating mycophenolic acid from the alkaline aqueous solution of step a).

14. A process as claimed in claim 13, wherein the alkaline acqueous solution comprises an organic or inorganic base.

15. A process as claimed in claim 14, wherein the inorganic base is selected from sodium hydroxide, potassium hydroxide, sodium carbonate, potassium carbonate, sodium bicarbonate and potassium bicarbonate.

16. (canceled)

17. A process as claimed in any one of the claims 13, wherein step b) occurs is accomplished by acidification.

18. A process as claimed in claim 1, wherein the salt is sodium mycophenolate.

19. A process as claimed in claim 18, further comprising:
f) dissolving the mycophenolic acid in a solvent selected from the group consisting of sulfolane, dimethylacetamide, dimethylsufoxide, N-methylpyrrolidone, acetonitrile or mixture thereof;
g) adding a source of sodium ion to form sodium mycophenolate; and
h) isolating sodium mycophenolate in M2 form.

20. A process as claimed in claim 19, wherein the source of sodium ion is selected from sodium methoxide, and sodium hydroxide.

21. A process as claimed in claim 1, wherein the mycophenolic acid derivative is mycophenolate mofetil.

22. A process as claimed in claim 21, wherein the process for mycophenolate mofetil further comprising the steps of:
f) reacting mycophenolic acid or its salt with a compound of formula IV or its salt to obtain mycophenolate mofetil; 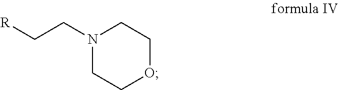
and
g) isolating mycophenolate mofetil from the reaction mixture.

23. A process as claimed in claim 22, wherein the mycophenolic acid salt is sodium and potassium salt.

24. A process for preparation of as claimed in claim 22, wherein wherein step f) occurs in presence of a base.

25. A process as claimed in claim 21, wherein preparation of mycophenolate mofetil comprising reacting mycophenolic acid with 2-(4-morpholinyl)ethanol in presence of molecular sieves.

26. A process as claimed in claim 25, wherein the reaction is in presence of a solvent.

27. A process as claimed in claim 5, wherein the chemical agent is N-methyl-N′-nitro-N-nitroso-guanidine, ethyl methyl sulfonate, clofibrate, and/or methyl viologen.

28. A process as claimed in claim 13, furthering comprising filtering the alkaline aqueous solution prior to precipitating the mycophenolic acid.
